(19) United States
(12) Patent Application Publication
(10) Pub. No.: US 2017/0360853 A1
(43) Pub. Date: Dec. 21, 2017

(54) Gastro-Intestinal Biomarkers for Diagnosis and Therapies of Proteinopathies

(71) Applicant: ECOLE POLYTECHNIQUE FEDERALE DE LAUSANNE (EPFL), Lausanne (CH)

(72) Inventors: Theo Lasser, Denges (CH); Tristan Bolmont, Saint-Sulpice (CH); Taoufiq Harach, Chavannes-près-Renens (CH); Walid Hanna, Lausanne (CH); Frida Fåk, Lund (SE); Nittaya Marungruang, Malmö (SE)

(21) Appl. No.: 15/541,045

(22) PCT Filed: Dec. 23, 2015
(86) PCT No.: PCT/IB2015/059945

§ 371 Date: Jun. 30, 2017

(30) Foreign Application Priority Data
Jan. 7, 2015 (IB) PCT/IB2015/050127
May 27, 2015 (IB) PCT/IB2015/053957

(51) Int. Cl. A61K 35/747; G01N 33/50; G01N 33/53

(52) U.S. Cl. CPC A61K 35/747; G01N 33/53; G01N 33/5008; G01N 2500/10; G01N 2333/90

(57) ABSTRACT

Pharmaceutical composition for the treatment of proteinopathies comprising products which are able to stimulate growth or inhibition of at least one of Odoribacter, Oscillospira, Dehalobacterium, Alistipes, Parabacteroides, Lactobacillus and Sutterella, Firmicutes, Bacteroidetes, Allobaculum and Akkermansia bacteria population in the gut of individuals.

### FIELD OF THE INVENTION

The present invention relates to the modulation of gastro-intestinal microflora as cure of proteinopathies in general and Alzheimer disease, in particular. The treatment of proteinopathies with a dietary intervention or a specifically adapted nutrition will include the administration of a probiotic and/or a prebiotic mixture or a pharmaceutical compound for a selective modulation of the gastro-intestinal microflora.

### STATE OF THE ART

To date, there is no cure for this devastating neurodegenerative disorder. Both clinical and epidemiological evidence suggest that, among other non-genetic factors, modification of lifestyle factors such as nutrition may prove crucial to the development of AD neuropathology. Higher fat intake and excess body weight increase the risk of AD, which is consistent with current epidemiological studies suggesting that obesity and diabetes are associated with increased risk of developing AD. One potential target against these metabolic diseases is the gut microflora community or microbiota. Diet is a well-characterized modulator of intestinal microflora. However, daily meal ingestion constitutes an uncontrolled way to modulate microflora.

In a living organism, proteins are involved in almost every biological process. They are synthesized on ribosomes as linear chains of amino acids from information encoded within the cellular DNA. In order to perform their biological function these chains of amino acids must fold into the native three-dimensional conformation that are characteristic of the individual proteins. How and whether primarily its amino acid sequence and the cellular environment surrounding the amino acid chain influence a protein folds. Mutations, abnormal physiological concentrations, coupled with prolonged time and certain biochemical conditions are thought to destabilize the native three-dimensional state, or divert soluble proteins from their normal folding pathway, often leading to their aggregation into stable insoluble amyloid deposits. Numerous degenerative diseases arise due to the buildup of insoluble misfolded protein deposits. These proteinopathies include neurological disorders such as Alzheimer's disease, Parkinson's disease, Huntington's disease, and also bovine spongiform encephalopathy and its human equivalent Creutzfeldt-Jakob disease, in addition to diverse systemic amyloidosis (Table 1).

TABLE 1
List of diseases resulting from amyloid formation

| | Disease | Protein Involved |
|---|---|---|
| | Alzheimer's disease | Amyloid β-peptide |
| | Spongiform encephalopathies | Prion protein |
| | Hereditary cerebral hemorrhage with | Amyloid β-peptide or crystatin C |
| | amyloidosis | |
| | Type II diabetes | Amylin (Islet amyloid polypeptide) |
| | Medullary carcinoma of the thyroid | Procalcitonin |
| | Atrial amyloidosis | Atrial natriuretic factor Primary |
| | | systemic |
| Systemic extracellular | Primary systemic amyloidosis | Intact Ig light chains or fragments |
| amyloidosis | Secondary systemic amyloidosis | Fragments of serum amyloid A |
| | | protein |
| | Familial Mediterranean fever | Fragments of serum amyloid A |
| | | protein |
| | Familial amyloidotic polyneuropathy1 | Mutant transthyretin and |
| | | fragments |
| | Senile systemic amyloidosis | Wild-type transthyretin and |
| | | fragments |
| | Familial amyloidotic polyneuropathy II | Fragments of apolipoprotein A-1 |
| | Haemodialysis-related amyloidosis | 2-Microglobulin |
| | Finnish hereditary amyloidosis | Fragments of mutant gelsolin |
| | Lysozyme amyloidosis | Full-length mutant lysozyme |
| | Insulin-related amyloid | Full-length insulin |
| | Fibrinogen α-chain amyloidosis | Fibrinogen α-chain variants |
| Intracellular amyloidosis | Alzheimer's disease | Amyloid β-peptide, Tau |
| | Frontotemporal dementia with | Tau |
| | parkinsonism | |
| | Parkinson's disease; dementia with Lewy | α-synuclein |
| | bodies | |
| | Creutzfeldt-Jakob disease | Prion protein |
| | Amyotrophic lateral sclerosis | Superoxide dismutase |
| | Polyglutamine expansion diseases | Long glutamine stretches within |
| | | proteins |

No sequence or structural similarities are apparent between any of the proteins that display the ability to form amyloids. Despite these differences, the fibrils formed by different polypeptides share a number of structural characteristics. For example, X-ray fiber diffraction studies indicate that the peptide backbone of the fibers adopts a cross β-sheet structure. In this structure, the individual β-sheets are oriented perpendicular to the long axis of the fiber, while the hydrogen bonds are oriented parallel with the long axis of the fiber. Amyloid fibers are also resistant to proteolysis, and display characteristic wavelength dependent birefringence when stained with the histological dye Congo red and seen under polarized light. There are also striking similarities in the aggregation of many misfolded proteins, even if their propensity to aggregate can vary markedly between different sequences. Amyloid fibrils are thought to form through self-assembly of protein monomers via a nucleation-dependent pathway initiated in partially denatured states of amyloidogenic proteins. However the folding or miss-folding process is not yet completely deciphered.

Alzheimer's disease (AD) is one of these protein conformational diseases and the leading cause of dementia in the Western world. Postmortem, it is characterized by two major neuropathological features:
extracellular deposition of A β peptides and
Intracellular aggregates of neurofibrillary lesions made of hyperphosphorylated tau proteins.

The observation that rare, early-onset familial forms of AD are caused by mutations in the amyloid precursor protein (APP), presenilin-1 (PS1), or presenilin-2 (PS2) gene, all of which increase the production of A β, led to the so-called A β amyloid cascade hypothesis. This hypothesis proposes that the aggregation of polymerized forms of A β in soluble multimeric and/or insoluble senile plaque deposits in the brain is an early and critical event that triggers a cascade of pathological events leading to hyperphosphorylation and somatodendritic segregation of tau, formation of neurofibrillary lesions, neuroinflammation, degeneration of brain cells and, finally, dementia. To date, there is no cure for this devastating neurodegenerative disorder.

Both clinical and epidemiological evidence suggest that, among other non-genetic factors, modification of lifestyle factors such as nutrition may have a crucial impact on the development of AD neuropathology. Higher fat intake and excess body weight seems to increase the risk of AD, which is consistent with current epidemiological studies suggesting that obesity and diabetes are associated with increased risk of developing AD. One potential target against these metabolic diseases is the gut microflora community or microbiota. However, this interaction between microbiota and proteinopathies in general and AD in particular is so far not perceived as a route for future risk evaluation and therapy.

The human gastro-intestinal (GI) tract hosts billions of bacteria, which triggers many pleiotropic effects including the immune system homeostasis, food processing, production and secretion of vitamins, modification of bile acid and communication with the Central Nervous System (CNS). Perturbation of the microbial community in the gastro-intestinal tract may contribute to an impaired homeostasis associated with disease states, such as neurological disorders. As aforementioned, both genetic and environmental factors are important in the etiology of AD. A potentially important environmental factor is abnormal intestinal flora that often interacts with other factors such as intestinal permeability and transport of toxic substances. Again these findings and concepts point to a strong, but yet not exploited mean for the therapy or diagnosis of proteinopathies.

In summary, there is a need to provide an efficient therapy and diagnostic tools for treating proteinopathies, and AD in particular.

### SUMMARY OF THE INVENTION

The present invention is based on the surprising findings that, in contrast to mouse models with microbiota (or conventionally raised (CONV) mice), germ-free (GF) mice are protected against the metabolic syndrome that develops after consuming a high-fat diet, suggesting that the diet-induced modulation of gut microflora improve metabolic diseases such as obesity and diabetes. As the aforementioned diseases increase the risk of developing AD, modifications of gut microbiota beneficially influence AD neuropathology. To elucidate the role of microbiota on cerebral Aβ amyloidosis, the inventors compared GF mice at different stages of amyloid pathology with AD mice raised in a conventional environment.

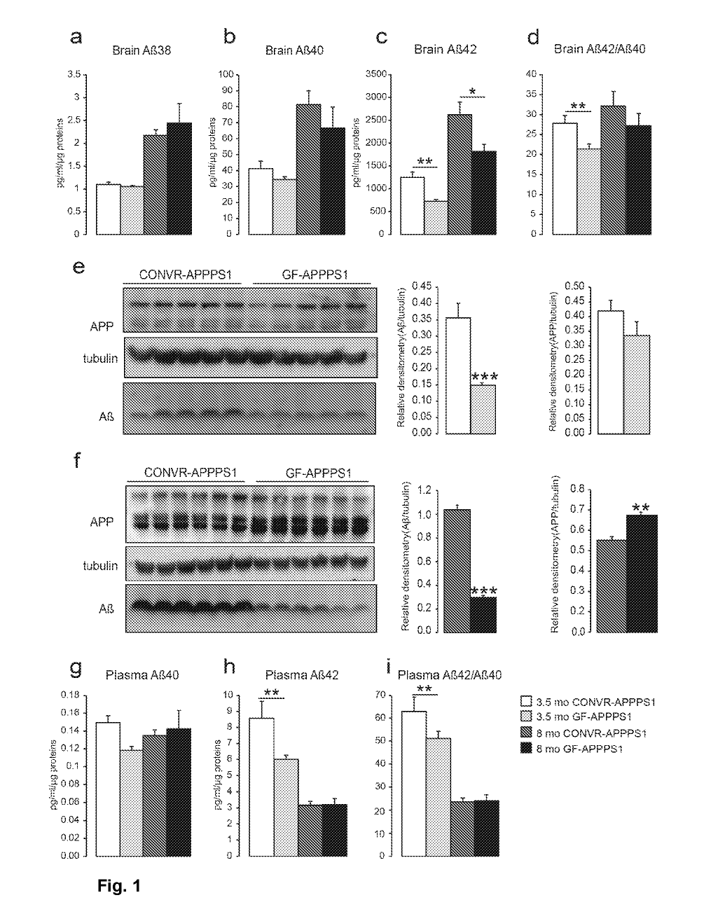

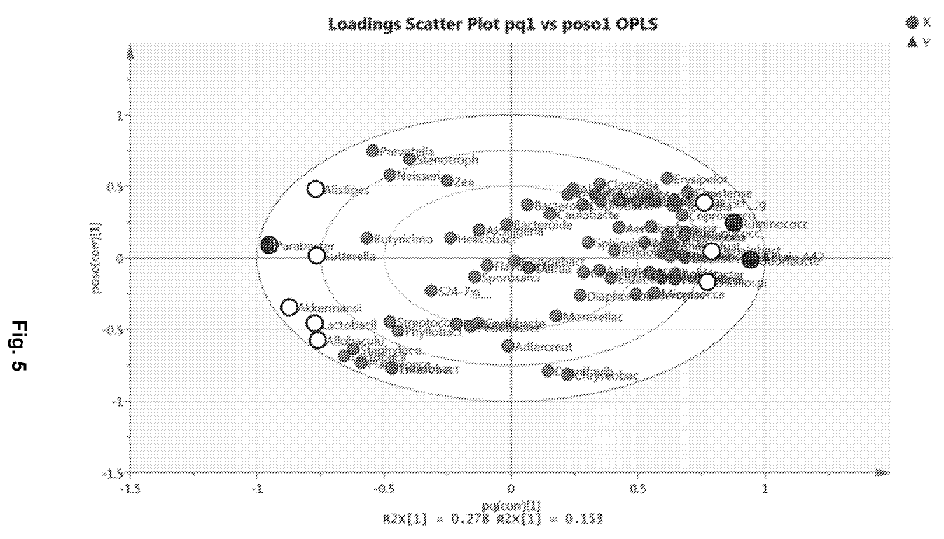

In GF mice, cerebral Aβ42 was significantly decreased in both young and aged animals, whereas Aβ40 levels remain unaffected compared with CONV mice. (See FIG. 1). Based on ELISA measurements, the inventors demonstrated that soluble A13 was significantly lower in both young and aged GF mice (See FIG. 1). In addition the inventors observed a substantial reduction in extracellular plaque density when comparing CONV-mice versus GF mice. These observations on plaque load are observed in parallel and correlate with the modulations of the microbiota spectrum and specifically in Firmicutes, Bacteroidetes, Allobaculum and Akkermansia bacteria populations (See FIG. 5). In addition, both soluble Aβ and plaque load are positively correlated with Odoribacter, Oscillospira and Dehalobacterium, whereas Alistipes, Parabacteroides, Lactobacillus and Sutterella are negatively correlated or anti-correlated with amyloid pathology.

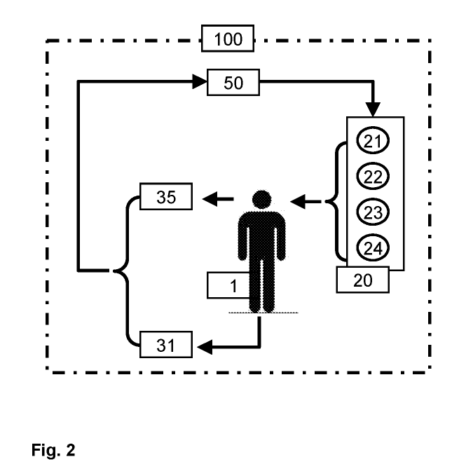

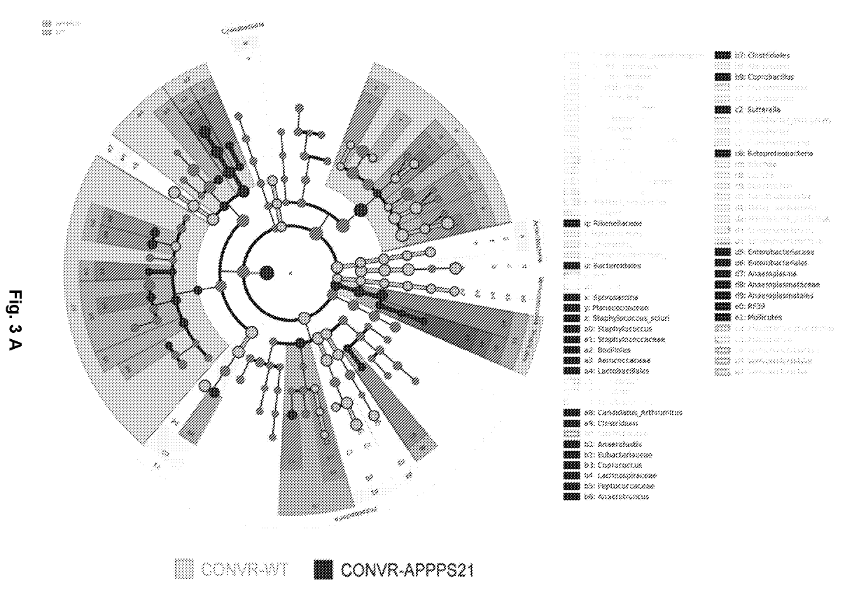

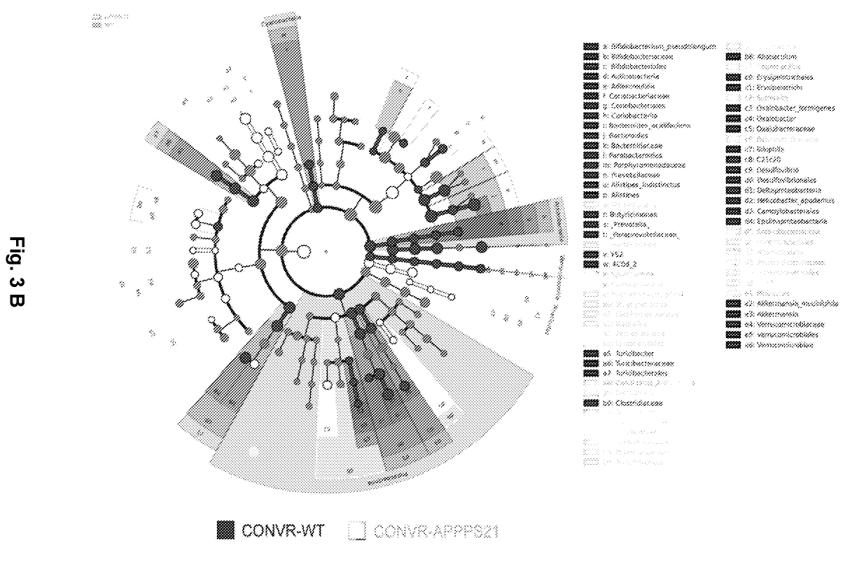

An objective of the invention is to provide a solution at least for the above-mentioned problems and advantages and improvements described herewith the following. (See FIG. 2)

Another objective of the invention relates to the characterization of bacterial populations of non-demented hosts versus AD individuals in order to identify differential properties and profile of a bacterial niche that can be used as therapeutic targets against proteinopathies, in particular but not exclusively AD. The differential properties (signatures) are identified using methods to profile the microbiota including screening of 16SrRNA genes by PCR and high-throughput methods such as pyrosequencing, allowing the identification of bacterial genes that are differentially represented in non-AD individuals versus AD patients.

Another objective of the present invention is to describe cures for proteinopathies, including AD by modulating the microbiota spectrum.

Another objective is to offer new putative drugs in the treatment of AD by targeting all microbial-modified and secreted products.

Those objectives are achieved with the products and methods defined in the independent claims.

Preferred embodiments of the invention are defined in the dependent claims. Additional advantages and improvements of this invention will be set forth in the description which follows and will become evident to those skilled in the art upon examination of the following or may learn from practice of this invention. The objects and advantages of this invention may be attained as particularly pointed out in the appended claims.

### DETAILED DESCRIPTION OF THE INVENTION

Referring to FIG. 1 an individual is diagnosed for his cognitive impairments induced by a proteinopathy (35) and/or by measuring its microbiota spectrum (31).

This microbiota spectrum (31) of a human individual contains at least one of Odoribacter, Oscillospira, Dehalobacterium, Alistipes, Parabacteroides, Lactobacillus and Sutterella, Firmicutes, Bacteroidetes, Allobaculum and Akkermansia bacteria population. The genomic DNA of microbiota is extracted from the feces i.e. the waste product originating from the gut of the individual. This microbiota spectrum (31) is assessed using profiling methods including screening of 16SrRNA genes by PCR and high-throughput methods such as pyrosequencing, allowing the identification of bacterial genes that are differentially represented in an individual. This allows determining a relative abundance of the at least one of Odoribacter, Oscillospira, Dehalobacterium, Alistipes, Parabacteroides, Lactobacillus and Sutterella, Firmicutes, Bacteroidetes, Allobaculum and Akkermansia bacteria population that is below or above a predetermined abundance or predetermined baseline.

Based on these findings an appropriate therapeutic decision is taken (50), which aims at a modulation of the of at least Odoribacter, Oscillospira, Dehalobacterium, Alistipes, Parabacteroides, Lactobacillus and Sutterella, Firmicutes, Bacteroidetes, Allobaculum and Akkermansia bacteria population by administrating probiotics (21), prebiotics (22), purified prebiotics (23) and/or antibiotics (24) as an isolated intake or a mixture or a combination of administrated means (20; comprising 21-24) or any mean to alter the microbiota spectrum for aiming a positive therapeutic result. This therapy (100) can be a single administration or consist in a repetitive administration until a positive therapeutic effect is achieved.

This invention describes in at least one aspect, a protein assay that includes protein extracted from the microbiota of a human individual to an assay that determines at least one protein indicative of Odoribacter, Oscillospira, Dehalobacterium, Alistipes, Parabacteroides, Lactobacillus and Sutterella, Firmicutes, Bacteroidetes, Allobaculum and Akkermansia bacteria population.

Products produced by Odoribacter, Oscillospira, Dehalobacterium, Alistipes, Parabacteroides, Lactobacillus and Sutterella, Firmicutes, Bacteroidetes, Allobaculum and Akkermansia bacteria including but not limited to proteins, carbohydrates, nucleic acids and/or lipids or more specifically short chain fatty acids, are used for modifying the microbiota in the gastrointestinal tract of an individual as part of a therapeutic regimen for treating AD.

In at least one aspect, a method of treating a human individual with proteinopathies, includes administering to the individual, bacteria-generated products including proteins, carbohydrates, nucleic acids and/or lipids or more specifically short chain fatty acids, to stimulate growth or inhibition of at least one of Odoribacter, Oscillospira, Dehalobacterium, Alistipes, Parabacteroides, Lactobacillus and Sutterella, Firmicutes, Bacteroidetes, Allobaculum and Akkermansia bacteria population in the gut of individuals.

In at least one aspect, a method of treating a human individual with AD, includes administering to the individual, bacteria-generated products including proteins, carbohydrates, nucleic acids and/or lipids or more specifically short chain fatty acids, to stimulate growth or inhibition of at least one of Odoribacter, Oscillospira, Dehalobacterium, Alistipes, Parabacteroides, Lactobacillus and Sutterella, Firmicutes, Bacteroidetes, Allobaculum and Akkermansia bacteria population in the gut of individuals.

This invention describes, a method of treating a human individual with proteinopathies, including the administration to the individual, prebiotics (proteins, carbohydrates, nucleic acids and/or lipids or more specifically short chain fatty acids), to stimulate growth or inhibition of at least one of Odoribacter, Oscillospira, Dehalobacterium, Alistipes, Parabacteroides, Lactobacillus and Sutterella, Firmicutes, Bacteroidetes, Allobaculum and Akkermansia bacteria population in the gut of individuals.

In at least one aspect, a method of treating a human individual with AD, includes administering to the individual, prebiotics (proteins, carbohydrates, nucleic acids and/or lipids or more specifically short chain fatty acids), to stimulate growth or inhibition of at least one of Odoribacter, Oscillospira, Dehalobacterium, Alistipes, Parabacteroides, Lactobacillus and Sutterella, Firmicutes, Bacteroidetes, Allobaculum and Akkermansia bacteria population in the gut of individuals.

This invention describes, a method of treating a human individual with proteinopathies, including the administration to the individual, probiotics including Odoribacter, Oscillospira, Dehalobacterium, Alistipes, Parabacteroides, Lactobacillus and Sutterella, Firmicutes, Bacteroidetes, Allobaculum and Akkermansia to stimulate growth or inhibition of at least one of Odoribacter, Oscillospira, Dehalobacterium, Alistipes, Parabacteroides, Lactobacillus and Sutterella, Firmicutes, Bacteroidetes, Allobaculum and Akkermansia bacteria population in the gut of individuals.

This invention describes, a method of treating a human individual with AD including the administration to the individual, probiotics including Odoribacter, Oscillospira, Dehalobacterium, Alistipes, Parabacteroides, Lactobacillus and Sutterella, Firmicutes, Bacteroidetes, Allobaculum and Akkermansia to stimulate growth or inhibition of at least one of Odoribacter, Oscillospira, Dehalobacterium, Alistipes, Parabacteroides, Lactobacillus and Sutterella, Firmicutes, Bacteroidetes, Allobaculum and Akkermansia bacteria population in the gut of individuals.

This invention describes, a method of treating a human individual with proteinopathies, including the administration to the individual of pharmaceutical compound to stimulate growth or inhibition of at least one of Odoribacter, Oscillospira, Dehalobacterium, Alistipes, Parabacteroides, Lactobacillus and Sutterella, Firmicutes, Bacteroidetes, Allobaculum and Akkermansia bacteria population in the gut of individuals.

This invention describes, a method of treating a human individual with AD, including the administration to the individual of pharmaceutical compound to stimulate growth or inhibition of at least one of Odoribacter, Oscillospira, Dehalobacterium, Alistipes, Parabacteroides, Lactobacillus and Sutterella, Firmicutes, Bacteroidetes, Allobaculum and Akkermansia bacteria population in the gut of individuals.

### DETAILED DESCRIPTION OF FIGURES

Referring to FIG. 1 as the shows the cerebral and plasmatic soluble Aβ and the reduction of cerebral and plasmatic soluble Aβ levels in GF-APPPS1 transgenic mice. Levels of cerebral soluble Aβ38 (a), Aβ40 (b), Aβ42 (c) and ratio Aβ42/Aβ40 (d) as measured by ELISA in 3.5 (n=7) and 8 (n=7) month-old conventionally-raised (CONVR-APPPS1) and in comparison to germ free (GF-APPPS1) APPPS1 mice. Levels of Aβ are assessed by western blot in 3.5 month-old (n=5) (e) and in 8 month-old mice (n=6) (f). The relative densities in (e) are shown aside and indicate the relative density of (Aβ/tubulin) and (APP/tubulin) for CONV-APPPS1 in comparison to GF-APPPS1 mice. This teaching is equivalent in (f) but for 8-month old mice for both mice species. Plasmatic levels of soluble Aβ40 (g), Aβ42 (h) and ratio Aβ42/Aβ40 (i) measured by ELISA.

Referring to FIG. 2 an individual is diagnosed for his cognitive impairments induced by a proteinopathy (35) and/or by measuring its microbiota spectrum (31). An appropriate therapeutic decision is taken (50), which aims at modulating gut microbiota by administrating probiotics (21), prebiotics (22), purified prebiotics (23) and/or antibiotics (24) as an isolated intake or a mixture or a combination of administrated means (20; comprising 21-24). This therapy (100) can be a single administration or consist in a repetitive administration until a positive therapeutic effect is achieved.

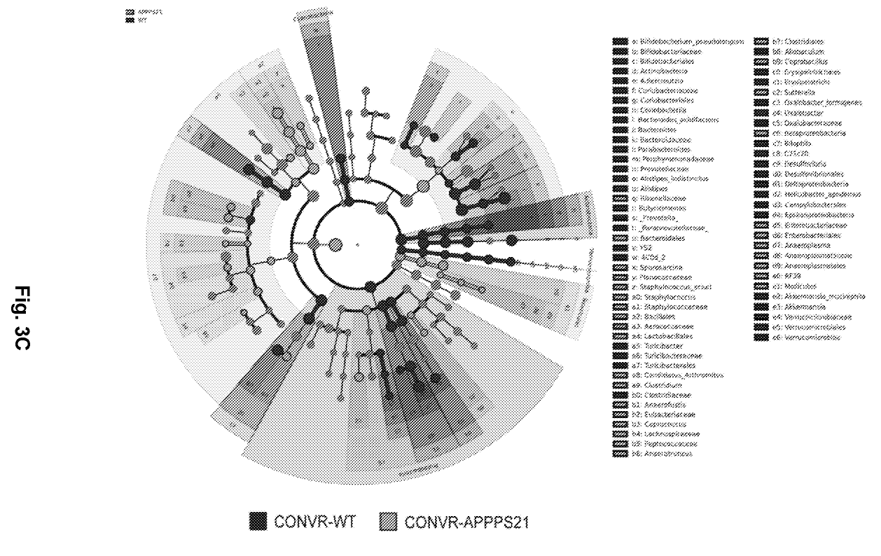

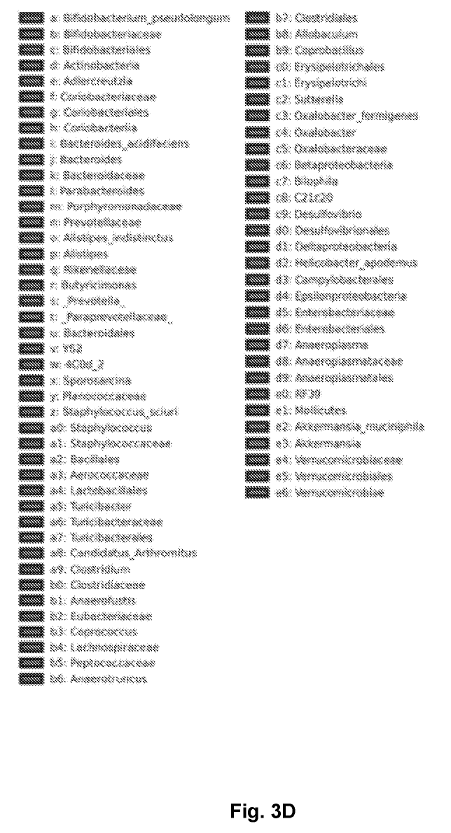

Referring to FIG. 3A to FIG. 3C are Cladograms generated with LDA Effect Size (LEfSE) analysis for 219,712 randomly selected sequences/sample illustrating enrichment of the phylum Bacteroidetes in APPPS21 (CONV) mice while WT mice show enriched in Firmicutes and Verrucomicrobia. The size of circles is proportionate to each taxon's mean relative abundance. At genus level, unclassified genera of S24-7 and Rikenellaceae were increased in APPPS21 mice, and Allobaculum and Akkermansia in WT mice. FIG. 3A-C shows cladograms, where FIG. 3A shows the relative abundance of gut-bacteria in conventially raised APPPS1 mice, FIG. 3B in WT mice and FIG. 3C the overlay for both mice in a full cladogram. FIG. 3D is the associated table for naming the bacteria. The tables in FIG. 3A name the bacteria for cladogram shown in FIG. 3A, similar teaching is valid for FIG. 3B.

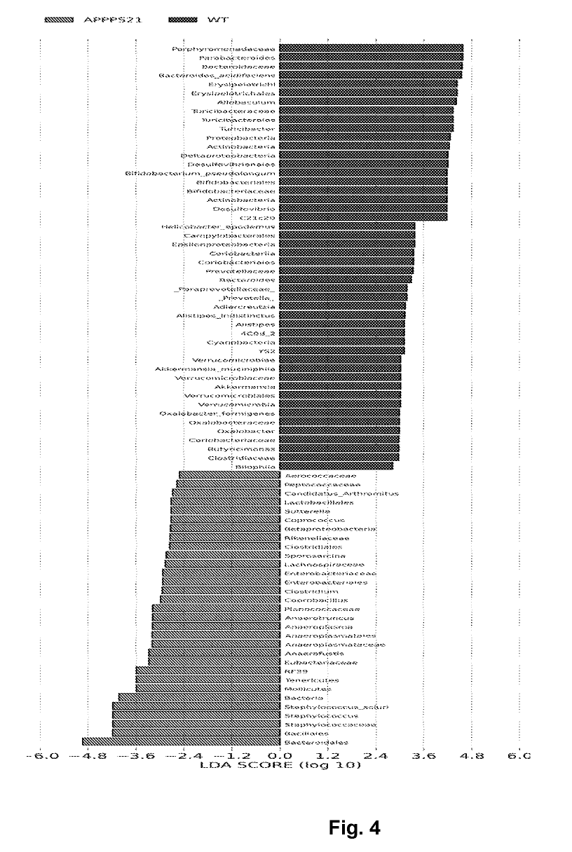

Referring to FIG. 4 as mean sequence relative abundance of gut microbiota in 8-months-old transgenic APPPS21 (n=7) and wild type (WT, n=6)) mice, at phylum (top left) and genus (middle left) level. LEfSE analysis revealed 8 differentially abundant phyla (bottom left) and 28 genera (right) (α=0.01) with an LDA score higher than 2 when comparing transgenic APPPS21 and WT mice.

Referring to FIG. 5 as an orthogonal partial least squares (OPLS) scatter plot of correlations between different gut microbial genera (x-variables) and cerebral soluble Aβ42 (Brain Aβ42; y-variable) in 8 months old conventional transgenic APPPS21 mice.

### Definitions Terms and Elements

Amyloid: Amyloids are insoluble fibrillar protein aggregates that share specific structural traits. Amyloids arise from at least 20 misfolded proteins and polypeptides present naturally in the body. These inappropriately folded structures alter their proper configuration such that they erroneously interact with one another or other cell components forming insoluble fibrils. Amyloids have been associated with the pathology of more than 20 serious human diseases in that, abnormal accumulation of amyloid fibrils in organs may lead to amyloidosis, and may play a role in various neurodegenerative disorders

Proteinaceous: Of, relating to, consisting of, resembling, or pertaining to protein, or pertaining to any material having a protein base.

Proteinopathy: Proteinopathy refers in medicine to a class of diseases in which certain proteins become structurally abnormal, and thereby disrupt the function of cells, tissues and organs of the body. Frequently the proteins fail to fold into their normal configuration; in this misfolded state, the proteins can become toxic in some way (a gain of toxic function) or they can lose their normal function. The proteinopathies (also known as proteinopathies, protein conformational disorders, or protein misfolding diseases) include diseases such as prion diseases, Alzheimer's disease, Parkinson's disease, type 2-diabetes, and a wide range of other central and peripheral disorders. The concept of proteopathy can trace its origins to the mid-19th century, when, in 1854, Rudolf Virchow coined the term amyloid (“starch-like”) to describe a substance in cerebral corpora amylacea that exhibited a chemical reaction resembling that of cellulose. In 1859, Friedreich and Kebulé demonstrated that, rather than consisting of cellulose, “amyloid” actually is rich in protein. Subsequent research has shown that many different proteins can form amyloid, and that all amyloids have in common birefringence in cross-polarized light after staining with the dye Congo Red, as well as a fibrillar ultrastructure when viewed with an electron microscope.

Proteolysis: Proteolysis is the directed degradation or digestion, of proteins by cellular enzymes called proteases or by intra-molecular digestion.

Seeded proteinopathies: Some proteins can be induced to form abnormal assemblies by exposure to the same or similar protein assembly that has folded into a disease-causing conformation, a process called ‘seeding’ or ‘permissive templating’. In this way, the disease state can be brought about in a susceptible host by the introduction of diseased tissue extract from an afflicted donor. The most known form of such inducible proteopathy is prion disease (e.g., Creutzfeldt Jakob), which can be transmitted by exposure of a host organism to purified prion protein in a disease-causing conformation. There is now evidence that other proteinopathies can be induced by a similar mechanism, including but not restricted to A13 amyloidosis, tauopathy and synucleinopathy. In all of these instances, an aberrant form of the protein itself appears to be the pathogenic agent. In some cases, the deposition of one type of protein can be experimentally induced by aggregated assemblies of other proteins that are rich in β-sheet structure, possibly because of structural complementarity of the protein molecules. There is also experimental evidence for cross-seeding between prion protein and Aβ or between tau and Aβ.

Microbiota/Microflora: The term “microbiota or microflora” refers to the whole microbial community found in the gastro-intestinal tract of a higher organism, including bacteria, archaea, yeasts, and various parasites.

Microflora modulation: Refers to a change in the representation of bacteria in a microbiological community of a particular individual.Microflora can be modulated bycompounds that induce the growth and/or activity of commensal microorganisms that contribute to the well-being of their host. These molecules called prebiotics are present in diet or in the gastro-intestinal tract and are typically but not exclusively non-digestible fibers compounds that stimulate the growth and/or activity of beneficial bacteria that colonize the large bowel by acting as substrate for them. Microflora can also be modulated by microorganisms. These live micro-organisms called probiotics, when administered adequately, confer a health benefit to the host. Gastro-intestinal microflora can be also modulated by antibiotics. These molecules refer to any substance produced by a living microorganism which is antagonistic to the growth and/or division of other living microorganisms. Nutritional approach leading to modulation of gut microbiota by a combination of probiotics and prebiotics are commonly referred as synbiotics. These molecules include carbohydrates, proteins, nucleic add and lipids.

Short chain fatty acids: are lipids which constitute a sub-group of fatty acids. They includes Acetic acid, Propionic acid, Isobutyric acid (2-methylpropanoic acid), Butyric acid, Isovaleric acid (3-methylbutanoic acid) and Valeric acid (pentanoic acid).

Features that allow specific modulation of microflora: Specifications that differentiate a defined microbial community from the rest of the microbes that may coexist in the same GI tract. These features include the identification and administration of any microbial-generated molecules associated with the aforementioned distinct microbial community. These molecules may be, but not exclusively proteins, carbohydrates, nucleic acids and/or lipids or more specifically short chain fatty acids.

Microbiome analysis: Techniques for characterizing the microbiome include use of nucleic acid and/or proteins. Nucleic acid analysis includes analysis of, DNA, RNA, mRNA, rRNA, and/or tRNA, and can be accomplished using, but not limited to pyrosequencing, qPCR, RT-qPCR, clone libraries, DGGE, T-RFLP, ARISA, micro arrays, FIFH, dot-blot hybridization, next generation sequencing, DNA mapping devices and any other DNA hybridization methods that will detect a specific sequence.

Proteome analysis: Protein analysis can be performed by 2-Dimensional Gel Electrophoresis, 2-Diminsional Difference Gel Electrophoresis (2D-DIGE), MALDI TOFMS, (2D-) LC-ESI-MS/MS, AQUA, and iTRAQ.

These characterizations are combined with statistical analysis (Linear discriminant analysis effect size (LEfSE, www.huttenhower.sph.harvard.edu/galaxy/)) to precisely determine the players within the microbiome. Rigorous bioinformatics analysis provides accurate characteristics and distributions of intestinal microflora between individuals. Changes in algorithms or data analysis are known and will in no case represent a novelty for the disclosed claims.

1. A pharmaceutical composition for the treatment of proteinopathies comprising products that stimulate growth or inhibition of at least one of Odoribacter, Oscillospira, Dehalobacterium, Alistipes, Parabacteroides, Lactobacillus and Sutterella, Firmicutes, Bacteroidetes, Allobaculum and Akkermansia bacteria population in a gut of an individual.

2. The pharmaceutical composition according to claim 1 wherein the products are bacteria-generated products including at least one of proteins, carbohydrates, nucleic acids, lipids, and short chain fatty acids.

3. The pharmaceutical composition according to claim 1 for the treatment of Alzheimer's Disease.

4. A method for determining an appropriate treatment of proteinopathies using a composition including products that stimulate growth or inhibition of at least one of Odoribacter, Oscillospira, Dehalobacterium, Alistipes, Parabacteroides, Lactobacillus and Sutterella, Firmicutes, Bacteroidetes, Allobaculum and Akkermansia bacteria population in a gut of an individual, the method comprising a step of:
determining a relative abundance of the at least one of Odoribacter, Oscillospira, Dehalobacterium, Ali stipes, Parabacteroides, Lactobacillus and Sutterella, Firmicutes, Bacteroidetes, Allobaculum and Akkermansia bacteria population that is below or above a predetermined abundance or predetermined baseline.

5. The method according to claim 4 further comprising a step of:
preparing a composition including products to stimulate growth or inhibition of at least one of Odoribacter, Oscillospira, Dehalobacterium, Alistipes, Parabacteroides, Lactobacillus and Sutterella, Firmicutes, Bacteroidetes, Allobaculum and Akkermansia bacteria population, to reach the baseline.
